(19) United States
(12) Patent Application Publication
(10) Pub. No.: US 2006/0270694 A1
(43) Pub. Date: Nov. 30, 2006

(54) JAK kinase inhibitors and their uses

(75) Inventor: Brian Wong, Los Altos, CA (US)

Correspondence Address: DECHERT LLP, P.O. BOX 10004, PALO ALTO, CA, 94303, US

(73) Assignee: Rigel Pharmaceuticals, Inc., South San Francisco, CA (US)

(21) Appl. No.: 11/416,652

(22) Filed: May 2, 2006

Related U.S. Application Data
(60) Provisional application No. 60/678,241, filed on May 3, 2005.

(51) Int. Cl. A61K 31/506; A61K 31/505

(52) U.S. Cl. 514/275

(57) ABSTRACT

The present disclosure provides 2,4-pyrimidinediamine compounds that selectively inhibit JAK kinase as compared to Syk kinase and various methods of using the JAK-selective compounds.

### 1. FIELD

The present disclosure relates generally to compounds that are selective inhibitors of JAK kinases, and methods of using the compounds and compositions in a variety of contexts.

### 2. BACKGROUND

Protein kinases constitute a large family of structurally related enzymes that are responsible for the control of a variety of signal transduction processes within cells (see e.g., Hardie and Hanks, The Protein Kinase Facts Book, I and II, Academic Press, San Diego, Calif., 1995). Protein kinases are thought to have evolved from a common ancestral gene due to the conservation of their structure and catalytic function. Almost all kinases contain a similar 250/300 amino acid catalytic domain. The kinases may be categorized into families by the substrates they phosphorylate (e.g., protein-tyrosine, protein-serine/threonine, lipids, etc.). Sequence motifs have been identified that generally correspond to each of these families (see, e.g., Hanks & Hunter, 1995, FASEB J. 9:576-596; Knighton et al., 1991, Science 253:407-414; Hiles et al., 1992, Cell 70:419-429; Kunz et al., 1993, Cell 73:585-596; Garcia-Bustos et al., 1994, EMBO J. 13:2352-2361).

Many diseases are associated with abnormal cellular responses triggered by protein kinase-mediated events. These diseases include autoimmune diseases, inflammatory diseases, bone diseases, metabolic diseases, neurological and neurodegenerative diseases, cancer, cardiovascular diseases, allergies, asthma, alzheimer's disease and hormone-related diseases. As a consequence, there has been substantial efforts in medicinal chemistry to find inhibitors of protein kinases for use as therapeutic agents.

The Janus kinases (JAK) are a family of tyrosine kinases consisting of JAK1, JAK2, JAK3 and TYK2. The JAKS play a crucial role in cytokine signaling. The downstream substrates of the JAK family of kinases include the signal tranducer activator of transcription (STAT) proteins. JAK/STAT signaling has been implicated in the mediation of many abnormal immune responses such as allergies, asthma, autoimmune diseases such as transplant (allograft) rejection, rheumatoid arthritis, amyotrophic lateral sclerosis and multiple sclerosis, as well as in solid and hematologic malignancies such as leukemia and lymphomas. For a review of the pharmaceutical intervention of the JAK/STAT pathway see Frank, 1999, Mol. Med. 5:432:456 and Seidel et al., 2000, Oncogene 19:2645-2656.

JAK1, JAK2, and TYK2 are expressed ubiquitously, whereas JAK3 is expressed predominantly in hematopoietic cells. JAK3 binds exclusively to the common cytokine receptor gamma chain (Fcγ) and is activated by IL-2, IL-4, IL-7, IL-9 and IL-15.

JAK3 has been implicated in a variety of biological processes. For example, the proliferation and survival of murine mast cells induced by IL-4 and IL-9 have been shown to be dependent on JAK3- and gamma chain-signaling (Suzuki et al., 2000, Blood 96:2172-2180). JAK3 also plays a crucial role in IgE receptor-mediated mast cell degeneration responses (Malaviya et al., 1999, Biochem. Biophys. Res. Commun. 257:807-813), and inhibition of JAK3 kinase has been shown to prevent type I hypersensitivity reactions, including anaphylaxis (Malaviya et al., 1999, J. Biol. Chem. 274:27028-27038). JAK3 inhibition has also resulted in immune suppression for allograft rejection (Kirken, 2001, Transpl. Proc. 33:3268-3270).

JAK3 kinases have also been implicated in the mechanism involved in early and late stages of rheumatoid arthritis (Muller-Ladner et al., 2000, J. Immunal. 164:3894-3901); familial amyotrophic lateral sclerosis (Trieu et al., 2000, Biochem Biophys. Res. Commun. 267:22-25); leukemia (Sudbeck et al., 1999, Clin. Cancer Res. 5:1569-1582); mycosis fungoides, a form of T-cell lymphoma (Nielsen et al., 1997, Prac. Natl. Acad. Sci. USA 94:6764-6769); and abnormal cell growth (Yu et al., 1997, J. Immunal. 159:5206-5210; Catlett-Falcone et al., 1999, Immunity 10:105-115).

Syk kinase is also a tyrosine kinase, and is known to play a critical role in Fcγ receptor signaling, as well as in other signaling cascades, such as those involving B-Cell receptor signaling (Tumer et al., 2000, Immunology Today 21:148-154) and integrins beta(1), beta (2) and beta (3) in neutrophils (Mocsavi et al., 2002, Immunity 16:547-558). For example, Syk kinase plays a pivotal role in high affinity IgE receptor signaling in mast cells that leads to activation and subsequent release of multiple chemical mediators that trigger allergic attacks. However, unlike the JAK kinases, which help regulate the pathways involved in delayed, or cell-mediated Type IV hypersensitivity reactions, Syk kinase helps regulate the pathways involved in immediate IgE-mediated, Type I hypersensitivity reactions.

Given the important roles of JAK kinases, the availability of compounds that inhibit these kinases would be desirable. Moreover, the availability of compounds that selectively inhibit JAK kinases as compared to Syk kinase would also be desirable.

### 3. SUMMARY

It has now been discovered that certain 2,4-pyrimdinediamine compounds are potent inhibitors of JAK kinases. The compounds are also selective for JAK kinases as compared to Syk kinase. Thus, in one aspect, the present disclosure provides 2,4-pyrimdinediamine compounds that are potent and selective inhibitors of JAK kinases. Some embodiments of these compounds are also inhibitors of Syk kinase, whereas some embodiments of these compounds do not appreciably inhibit Syk kinases (i.e., exhibit IC₅₀s of greater than about 20 μM in cellular and/or biochemical assays designed to test for Syk inhibition). Embodiments that inhibit Syk kinase are at least 10-fold more potent against a JAK kinase than Syk kinase, as measured in cellular and/or biochemical assays that will be described in more detail below. Many embodiments are at least about 100-fold, more potent against a JAK kinase than Syk kinase.

All of the compounds described herein, whether active or inactive against Syk kinase, are potent inhibitors of a JAK kinase, exhibiting IC₅₀s of less than about 5 μM in cellular and/or biochemical assays designed to test for JAK kinase inhibition.

The degree to which any compound can inhibit Syk kinase and still be considered “selective” for a JAK can vary, and will depend, in part, on the potency of the particular compound against a JAK kinase. Compounds are considered JAK selective with respect to Syk if they are at least about 10-fold more potent against a JAK kinase than Syk kinase (i.e., the ratios of their IC₅₀ (Syk): IC₅₀ (JAK) are at least about 10). In preferred embodiments, JAK selective compounds are at least about 100-fold more potent against a JAK kinase than Syk kinase. Embodiments of compounds that do not appreciably inhibit Syk kinases (i.e., have an IC₅₀ of ≧20 μM in the CHMC assay described in the Examples section or other Syk inhibition assay) are referred to herein as “highly JAK-selective.”

The JAK-selective inhibitory compounds are generally 2,4-pyrimdinediamine compounds that are substituted at the N2 nitrogen atom with an optionally substituted aryl or hetaroaryl group and at the N4 nitrogen atom with an optionally substituted alkyl, cycloalkyl, heteroalky, cycloharoalkyl, aryl or hetaroaryl group. Alternatively, the N4 nitrogen atom can be included in a saturated 4-8 membered ring. The pyrimidine ring can be unsubsituted at the 5-position, but is typically substituted with an electronegative group. In one specific embodiment, the electronegative group is selected from an aldehyde, a ketone, a carboxylic acid, and ester, nitro, cyano, trifluoromethyl, trifluoromethoxy, halo, bromo, chloro and fluoro. In another specific embodiment, the pyrimidine ring is substituted at the 5-position with a fluoro group. The 6-position of the pyrimidine ring is typically unsubstituted. Specific exemplary embodiments of JAK inhibitory compounds that are highly JAK-selective are illustrated in TABLE 1. Specific exemplary embodiments of compounds that are JAK-selective dual JAK-Syk inhibitors are illustrated in TABLE 2.

The compounds can be used to regulate, and in particular inhibit, JAK kinases in a variety of contexts. Thus, in another aspect, the present disclosure provides methods of regulating, and in particular inhibiting, a JAK kinase activity. The methods generally involve contacting a JAK kinase or a cell comprising a JAK kinase with an amount of a JAK-selective inhibitory compound described herein effective to regulate or inhibit a JAK kinase activity. In one embodiment, the JAK kinase is an isolated or recombinant JAK kinase. In another embodiment, the JAK kinase is an endogenous or recombinant JAK kinase expressed by a cell, for example a T-cell. The method may be practiced in in vitro contexts or in in vivo contexts as a therapeutic approach towards the treatment or prevention of diseases mediated, at least in part, by JAK kinase activity.

The JAK kinase regulated or inhibited can be any JAK kinase, or combination of JAK kinases. In some embodiment, the JAK kinase is JAK3 kinase, and the inhibitory compounds are selective for JAK3 kinase as compared to other members of the JAK kinase family (i.e., the IC₅₀ (JAK): IC₅₀ (JAK3) ratio is at least about 10).

As mentioned in the Background section, JAK3 kinase binds the common gamma chain of cytokinetic receptors. This common gamma chain, which is involved in both ligand binding and signal transduction, is a shared subunit of the multichain receptor for cytokines IL-2, IL-4, IL-7, IL-9, IL-15 and IL-21. Because the JAK3 kinase binds the common gamma chain of these receptors, the JAK inhibitory compounds described herein can be used to regulate, and in particular inhibit, these and other cytokine receptor signaling cascades which utilize the common gamma chain. Thus, in another aspect, the present disclosure provides methods of regulating, and in particular inhibiting, signal transduction cascades in which a JAK kinase plays a role, such as signal transduction cascades of cytokine receptors utilizing the common gamma chain, including, but not limited to, the IL-2, IL-4, IL-7, IL-9, IL-15 and IL-21 signal transduction cascades. The methods generally involve contacting a JAK-dependent receptor, or a cell expressing a JAK-dependent receptor, with an amount of JAK inhibitory compound effective to regulate or inhibit the signal transduction cascade. The methods may also be used to regulate, and in particular inhibit, downstream processes or cellular responses elicited by activation of the particular JAK-dependent signal transduction cascade. The methods may be practiced to regulate any signal transduction cascade where JAK kinase is now known or later discovered to play a role. The methods may be practiced in in vitro contexts or in in vivo contexts as a therapeutic approach towards the treatment or prevention of diseases characterized by, caused by or associated with activation of the JAK-dependent signal transduction cascade. Non-limited examples of such diseases are described below.

As mentioned in the Background section, the JAK kinases have been implicated and/or demonstrated to play a critical role in many disease pathways. As a consequence of their ability to inhibit JAK kinases, the compounds described herein can be used to treat or prevent virtually any disease that is mediated, at least in part, by a JAK kinase activity. Thus, in still another aspect, the present disclosure provides methods of treating or preventing diseases that are mediated, at least in part, by a JAK kinase. The methods may be practiced in animals in veterinary contexts, or in humans. The methods generally involve administering to an animal or human an amount of a JAK-inhibitory compound effective to treat or prevent the JAK-mediated disease. Examples of diseases that are mediated, at least in part, by JAK kinases that can be treated or prevented according to the methods include, but are not limited to, allergies, asthma, autoimmune diseases such as transplant rejection (e.g., kidney, heart, lung, liver, pancreas, skin, host versus graft reaction (HVGR), etc.), rheumatoid arthritis, and amyotrophic lateral sclerosis, multiple sclerosis, psoraiasis and Sjogren's syndrome, Type II inflammatory disease such as vascular inflammation (including vasculitis, ateritis, atherosclerosis and coronary artery disease), diseases of the central nervous system such as stroke, pulmonary diseases such as bronchitis obliterous and primary and primary pulmonary hypertension, delayed or cell-mediated, Type IV hypersensitivity and solid and hematologic malignancies such as leukemias and lyphomas.

Because the JAK-inhibitory compounds are selective for JAK kinases over Syk kinase, the JAK-inhibitory compounds described herein can be used in therapeutic and/or prophylactic regimens where Syk kinase inhibitors are ineffective. For example, they can be used in therapeutic and/or prophylactic regimens to treat and/or prevent diseases that are mediated, at least in part, by both a JAK kinase and Syk kinase, in patients that either fail to respond to treatment with Syk kinase inhibitory compounds, or that become nonresponsive to such compounds.

In other embodiments, the JAK-selective compounds can be used in therapeutic and/or phophylactic regimens to treat and/or prevent diseases that are mediated, at least in part, by a JAK kinase, but not Syk kinase.

In still other embodiments, the JAK-selective compounds can be used in conjunction or combination with Syk inhibitory compounds to treat and/or prevent diseases in which both Syk and a JAK pay a role. Specific, non-limiting, examples of such diseases include those described. The JAK-and Syk-inhibitory compounds can be administered together as combination therapy, or sequentially, one after the other. For example, the JAK and Syk inhibitory compounds can be administered at the same time, either together in the form of a combination composition, or at different times.

In some embodiments, the JAK inhibitory compounds are used adjunctively to existing therapies. For example, the initial, hyperacute rejection associated with allograft rejection in organ and tissue transplant patients is antibody dependent. However, acute rejection is mediated by T-cells. Since JAK kinases activate T-cells, the JAK inhibitory compounds described herein could be used as adjunct therapy to other allograft rejection treatments as an approach to ameliorate or prevent acute rejection. They can also be used to treat and/or prevent chronic rejection. In a specific embodiment, the acute rejection could be treated or prevented with a Syk inhibitory compound (and/or other conventional immunosuppressant) and the chronic rejection treated or prevented with a JAK-selective inhibitory compound described herein. In this context, the JAK-selective inhibitory compound could be administered adjunctively to the other therapies, as warranted.

While Syk kinase is involved in producing B-cells, JAK kinases are involved in the activation of T-cells. As a consequence, the JAK-selective inhibitory compounds are not expected to affect B-cell production, and are expected to be useful in the treatment or prevention of autoimmune disease that are mediated primarily by T-cells. Thus, in still another aspect, the present disclosure provides methods of treating or preventing autoimmune diseases that are mediated primarily by T-cells. The methods involve administering to an animal or human subject an amount of a JAK-selective inhibitory compound described herein effective to treat and/or prevent the T-cell mediated autoimmune disease. T-cell mediated autoimmune diseases that can be treated or prevented with the JAK-selective inhibitory compounds described herein include, but are not limited to multiple sclerosis, psoraiasis and Sjogren's syndrome.

### 4. BRIEF DESCRIPTION OF THE TABLES

TABLE 1 provides exemplary embodiments of JAK inhibitory compounds that are highly JAK-selective; and

TABLE 2 provides exemplary embodiments of JAK inhibitory compounds that are JAK-selective.

### 5. DETAILED DESCRIPTION OF THE PREFERRED EMBODIMENTS

5.1 Definitions

As used herein, the following terms are intended to have the following meanings:

“Alkyl” by itself or as part of another substituent refers to a saturated or unsaturated branched, straight-chain or cyclic monovalent hydrocarbon radical having the stated number of carbon atoms (i.e., C1-C6 means one to six carbon atoms) that is derived by the removal of one hydrogen atom from a single carbon atom of a parent alkane, alkene or alkyne. Typical alkyl groups include, but are not limited to, methyl; ethyls such as ethanyl, ethenyl, ethynyl; propyls such as propan-1-yl, propan-2-yl, cyclopropan-1-yl, prop-1-en-1-yl, prop-1-en-2-yl, prop-2-en-1-yl, cycloprop-1-en-1-yl; cycloprop-2-en-1-yl, prop-1-yn-1-yl, prop-2-yn-1-yl, etc.; butyls such as butan-1-yl, butan-2-yl, 2-methyl-propan-1-yl, 2-methyl-propan-2-yl, cyclobutan-1-yl, but-1-en-1-yl, but-1-en-2-yl, 2-methyl-prop-1-en-1-yl, but-2-en-1-yl, but-2-en-2-yl, buta-1,3-dien-1-yl, buta-1,3-dien-2-yl, cyclobut-1-en-1-yl, cyclobut-1-en-3-yl, cyclobuta-1,3-dien-1-yl, but-1-yn-1-yl, but-1-yn-3-yl, but-3-yn-1-yl, etc.; and the like. Where specific levels of saturation are intended, the nomenclature “alkanyl,” “alkenyl” and/or “alkynyl” is used, as defined below. In preferred embodiments, the alkyl groups are (C1-C6) alkyl.

“Alkanyl” by itself or as part of another substituent refers to a saturated branched, straight-chain or cyclic alkyl derived by the removal of one hydrogen atom from a single carbon atom of a parent alkane. Typical alkanyl groups include, but are not limited to, methanyl; ethanyl; propanyls such as propan-1-yl, propan-2-yl (isopropyl), cyclopropan-1-yl, etc.; butanyls such as butan-1-yl, butan-2-yl (sec-butyl), 2-methyl-propan-1-yl (isobutyl), 2-methyl-propan-2-yl (t-butyl), cyclobutan-1-yl, etc.; and the like. In preferred embodiments, the alkanyl groups are (C1-C6) alkanyl.

“Alkenyl” by itself or as part of another substituent refers to an unsaturated branched, straight-chain or cyclic alkyl having at least one carbon-carbon double bond derived by the removal of one hydrogen atom from a single carbon atom of a parent alkene. The group may be in either the cis or trans conformation about the double bond(s). Typical alkenyl groups include, but are not limited to, ethenyl; propenyls such as prop-1-en-1-yl, prop-1-en-2-yl, prop-2-en-1-yl, prop-2-en-2-yl, cycloprop-1-en-1-yl; cycloprop-2-en-1-yl; butenyls such as but-1-en-1-yl, but-1-en-2-yl, 2-methyl-prop-1-en-1-yl, but-2-en-1-yl, but-2-en-2-yl, buta-1,3-dien-1-yl, buta-1,3-dien-2-yl, cyclobut-1-en-1-yl, cyclobut-1-en-3-yl, cyclobuta-1,3-dien-1-yl, etc.; and the like. In preferred embodiments, the alkenyl group is (C2-C6) alkenyl.

“Alkynyl” by itself or as part of another substituent refers to an unsaturated branched, straight-chain or cyclic alkyl having at least one carbon-carbon triple bond derived by the removal of one hydrogen atom from a single carbon atom of a parent alkyne. Typical alkynyl groups include, but are not limited to, ethynyl; propynyls such as prop-1-yn-1-yl, prop-2-yn-1-yl, etc.; butynyls such as but-1-yn-1-yl, but-1-yn-3-yl, but-3-yn-1-yl, etc.; and the like. In preferred embodiments, the alkynyl group is (C2-C6) alkynyl.

“Alkyldiyl” by itself or as part of another substituent refers to a saturated or unsaturated, branched, straight-chain or cyclic divalent hydrocarbon group having the stated number of carbon atoms (i.e., C1-C6 means from one to six carbon atoms) derived by the removal of one hydrogen atom from each of two different carbon atoms of a parent alkane, alkene or alkyne, or by the removal of two hydrogen atoms from a single carbon atom of a parent alkane, alkene or alkyne. The two monovalent radical centers or each valency of the divalent radical center can form bonds with the same or different atoms. Typical alkyldiyl groups include, but are not limited to, methandiyl; ethyldiyls such as ethan-1,1-diyl, ethan-1,2-diyl, ethen-1,1-diyl, ethen-1,2-diyl; propyldiyls such as propan-1,1-diyl, propan-1,2-diyl, propan-2,2-diyl, propan-1,3-diyl, cyclopropan-1,1-diyl, cyclopropan-1,2-diyl, prop-1-en-1,1-diyl, prop-1-en-1,2-diyl, prop-2-en-1,2-diyl, prop-1-en-1,3-diyl, cycloprop-1-en-1,2-diyl, cycloprop-2-en-1,2-diyl, cycloprop-2-en-1,1-diyl, prop-1-yn-1,3-diyl, etc.; butyldiyls such as, butan-1,1-diyl, butan-1,2-diyl, butan-1,3-diyl, butan-1,4-diyl, butan-2,2-diyl, 2-methyl-propan-1,1-diyl, 2-methyl-propan-1,2-diyl, cyclobutan-1,1-diyl; cyclobutan-1,2-diyl, cyclobutan-1,3-diyl, but-1-en-1,1-diyl, but-1-en-1,2-diyl, but-1-en-1,3-diyl, but-1-en-1,4-diyl, 2-methyl-prop-1-en-1,1-diyl, 2-methanylidene-propan-1,1-diyl, buta-1,3-dien-1,1-diyl, buta-1,3-dien-1,2-diyl, buta-1,3-dien-1,3-diyl, buta-1,3-dien-1,4-diyl, cyclobut-1-en-1,2-diyl, cyclobut-1-en-1,3-diyl, cyclobut-2-en-1,2-diyl, cyclobuta-1,3-dien-1,2-diyl, cyclobuta-1,3-dien-1,3-diyl, but-1-yn-1,3-diyl, but-1-yn-1,4-diyl, buta-1,3-diyn-1,4-diyl, etc.; and the like. Where specific levels of saturation are intended, the nomenclature alkanyldiyl, alkenyldiyl and/or alkynyldiyl is used. Where it is specifically intended that the two valencies are on the same carbon atom, the nomenclature “alkylidene” is used. In preferred embodiments, the alkyldiyl group is (C1-C6) alkyldiyl. Also preferred are saturated acyclic alkanyldiyl groups in which the radical centers are at the terminal carbons, e.g., methandiyl (methano); ethan-1,2-diyl (ethano); propan-1,3-diyl (propano); butan-1,4-diyl (butano); and the like (also referred to as alkylenos, defined infra).

“Alkyleno” by itself or as part of another substituent refers to a straight-chain saturated or unsaturated alkyldiyl group having two terminal monovalent radical centers derived by the removal of one hydrogen atom from each of the two terminal carbon atoms of straight-chain parent alkane, alkene or alkyne. The locant of a double bond or triple bond, if present, in a particular alkyleno is indicated in square brackets. Typical alkyleno groups include, but are not limited to, methano; ethylenos such as ethano, etheno, ethyno; propylenos such as propano, prop[1]eno, propa[1,2]dieno, prop[1]yno, etc.; butylenos such as butano, but[1]eno, but[2]eno, buta[1,3]dieno, but[1]yno, but[2]yno, buta[1,3]diyno, etc.; and the like. Where specific levels of saturation are intended, the nomenclature alkano, alkeno and/or alkyno is used. In preferred embodiments, the alkyleno group is (C1-C6) or (C1-C3) alkyleno. Also preferred are straight-chain saturated alkano groups, e.g., methano, ethano, propano, butano, and the like.

“Heteroalkyl,” Heteroalkanyl,” Heteroalkenyl,” Heteroalkynyl,” Heteroalkyldiyl” and “Heteroalkyleno” by themselves or as part of another substituent refer to alkyl, alkanyl, alkenyl, alkynyl, alkyldiyl and alkyleno groups, respectively, in which one or more of the carbon atoms are each independently replaced with the same or different heteratoms or heteroatomic groups. Typical heteroatoms and/or heteroatomic groups which can replace the carbon atoms include, but are not limited to, —O—, —S—, —S—O—, —NR′—, —PH—, —S(O)—, —S(O)₂—, —S(O)NR′—, —S(O)₂NR′—, and the like, including combinations thereof, where each R′ is independently hydrogen or (C1-C6) alkyl.

“Cycloalkyl” and “Heterocycloalkyl” by themselves or as part of another substituent refer to cyclic versions of “alkyl” and “heteroalkyl” groups, respectively. For heteroalkyl groups, a heteroatom can occupy the position that is attached to the remainder of the molecule. Typical cycloalkyl groups include, but are not limited to, cyclopropyl; cyclobutyls such as cyclobutanyl and cyclobutenyl; cyclopentyls such as cyclopentanyl and cyclopentenyl; cyclohexyls such as cyclohexanyl and cyclohexenyl; and the like. Typical heterocycloalkyl groups include, but are not limited to, tetrahydrofuranyl (e.g., tetrahydrofuran-2-yl, tetrahydrofuran-3-yl, etc.), piperidinyl (e.g., piperidin-1-yl, piperidin-2-yl, etc.), morpholinyl (e.g., morpholin-3-yl, morpholin-4-yl, etc.), piperazinyl (e.g., piperazin-1-yl, piperazin-2-yl, etc.), and the like.

“Acyclic Heteroatomic Bridge” refers to a divalent bridge in which the backbone atoms are exclusively heteroatoms and/or heteroatomic groups. Typical acyclic heteroatomic bridges include, but are not limited to, —O—, —S—, —S—O—, —NR′—, —PH—, —S(O)—, —S(O)₂—, —S(O)NR′—, —S(O)₂NR′—, and the like, including combinations thereof, where each R′ is independently hydrogen or (C1-C6) alkyl.

“Parent Aromatic Ring System” refers to an unsaturated cyclic or polycyclic ring system having a conjugated π electron system. Specifically included within the definition of “parent aromatic ring system” are fused ring systems in which one or more of the rings are aromatic and one or more of the rings are saturated or unsaturated, such as, for example, fluorene, indane, indene, phenalene, tetrahydronaphthalene, etc. Typical parent aromatic ring systems include, but are not limited to, aceanthrylene, acenaphthylene, acephenanthrylene, anthracene, azulene, benzene, chrysene, coronene, fluoranthene, fluorene, hexacene, hexaphene, hexalene, indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, tetrahydronaphthalene, triphenylene, trinaphthalene, and the like, as well as the various hydro isomers thereof.

“Aryl” by itself or as part of another substituent refers to a monovalent aromatic hydrocarbon group having the stated number of carbon atoms (i.e., C5-C15 means from 5 to 15 carbon atoms) derived by the removal of one hydrogen atom from a single carbon atom of a parent aromatic ring system. Typical aryl groups include, but are not limited to, groups derived from aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene, and the like, as well as the various hydro isomers thereof. In preferred embodiments, the aryl group is (C5-C15) aryl, with (C5-C10) being even more preferred. Particularly preferred aryls are cyclopentadienyl, phenyl and naphthyl.

“Arylaryl” by itself or as part of another substituent refers to a monovalent hydrocarbon group derived by the removal of one hydrogen atom from a single carbon atom of a ring system in which two or more identical or non-identical parent aromatic ring systems are joined directly together by a single bond, where the number of such direct ring junctions is one less than the number of parent aromatic ring systems involved. Typical arylaryl groups include, but are not limited to, biphenyl, triphenyl, phenyl-naphthyl, binaphthyl, biphenyl-naphthyl, and the like. Where the number of carbon atoms in an arylaryl group are specified, the numbers refer to the carbon atoms comprising each parent aromatic ring. For example, (C5-C15) arylaryl is an arylaryl group in which each aromatic ring comprises from 5 to 15 carbons, e.g., biphenyl, triphenyl, binaphthyl, phenylnaphthyl, etc. Preferably, each parent aromatic ring system of an arylaryl group is independently a (C5-C15) aromatic, more preferably a (C5-C10) aromatic. Also preferred are arylaryl groups in which all of the parent aromatic ring systems are identical, e.g., biphenyl, triphenyl, binaphthyl, trinaphthyl, etc.

“Biaryl” by itself or as part of another substituent refers to an arylaryl group having two identical parent aromatic systems joined directly together by a single bond. Typical biaryl groups include, but are not limited to, biphenyl, binaphthyl, bianthracyl, and the like. Preferably, the aromatic ring systems are (C5-C15) aromatic rings, more preferably (C5-C10) aromatic rings. A particularly preferred biaryl group is biphenyl.

“Arylalkyl” by itself or as part of another substituent refers to an acyclic alkyl group in which one of the hydrogen atoms bonded to a carbon atom, typically a terminal or sp³ carbon atom, is replaced with an aryl group. Typical arylalkyl groups include, but are not limited to, benzyl, 2-phenylethan-1-yl, 2-phenylethen-1-yl, naphthylmethyl, 2-naphthylethan-1-yl, 2-naphthylethen-1-yl, naphthobenzyl, 2-naphthophenylethan-1-yl and the like. Where specific alkyl moieties are intended, the nomenclature arylalkanyl, arylakenyl and/or arylalkynyl is used. In preferred embodiments, the arylalkyl group is (C6-C21) arylalkyl, e.g., the alkanyl, alkenyl or alkynyl moiety of the arylalkyl group is (C1-C6) and the aryl moiety is (C5-C15). In particularly preferred embodiments the arylalkyl group is (C6-C13), e.g., the alkanyl, alkenyl or alkynyl moiety of the arylalkyl group is (C1-C3) and the aryl moiety is (C5-C 10).

“Parent Heteroaromatic Ring System” refers to a parent aromatic ring system in which one or more carbon atoms are each independently replaced with the same or different heteroatoms or heteroatomic groups. Typical heteroatoms or heteroatomic groups to replace the carbon atoms include, but are not limited to, N, NH, P, O, S, S(O), S(O)₂, Si, etc. Specifically included within the definition of “parent heteroaromatic ring systems” are fused ring systems in which one or more of the rings are aromatic and one or more of the rings are saturated or unsaturated, such as, for example, benzodioxan, benzofuran, chromane, chromene, indole, indoline, xanthene, etc. Also included in the definition of “parent heteroaromatic ring system” are those recognized rings that include common substituents, such as, for example, benzopyrone and 1-methyl-1,2,3,4-tetrazole. Specifically excluded from the definition of “parent heteroaromatic ring system” are benzene rings fused to cyclic polyalkylene glycols such as cyclic polyethylene glycols. Typical parent heteroaromatic ring systems include, but are not limited to, acridine, benzimidazole, benzisoxazole, benzodioxan, benzodioxole, benzofuran, benzopyrone, benzothiadiazole, benzothiazole, benzotriazole, benzoxaxine, benzoxazole, benzoxazoline, carbazole, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, and the like.

“Heteroaryl” by itself or as part of another substituent refers to a monovalent heteroaromatic group having the stated number of ring atoms (e.g., “5-14 membered” means from 5 to 14 ring atoms) derived by the removal of one hydrogen atom from a single atom of a parent heteroaromatic ring system. Typical heteroaryl groups include, but are not limited to, groups derived from acridine, benzimidazole, benzisoxazole, benzodioxan, benzodiaxole, benzofuran, benzopyrone, benzothiadiazole, benzothiazole, benzotriazole, benzoxazine, benzoxazole, benzoxazoline, carbazole, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, and the like, as well as the various hydro isomers thereof. In preferred embodiments, the heteroaryl group is a 5-14 membered heteroaryl, with 5-10 membered heteroaryl being particularly preferred.

“Heteroaryl-Heteroaryl” by itself or as part of another substituent refers to a monovalent heteroaromatic group derived by the removal of one hydrogen atom from a single atom of a ring system in which two or more identical or non-identical parent heteroaromatic ring systems are joined directly together by a single bond, where the number of such direct ring junctions is one less than the number of parent heteroaromatic ring systems involved. Typical heteroaryl-heteroaryl groups include, but are not limited to, bipyridyl, tripyridyl, pyridylpurinyl, bipurinyl, etc. Where the number of atoms are specified, the numbers refer to the number of atoms comprising each parent heteroaromatic ring systems. For example, 5-15 membered heteroaryl-heteroaryl is a heteroaryl-heteroaryl group in which each parent heteroaromatic ring system comprises from 5 to 15 atoms, e.g., bipyridyl, tripuridyl, etc. Preferably, each parent heteroaromatic ring system is independently a 5-15 membered heteroaromatic, more preferably a 5-10 membered heteroaromatic. Also preferred are heteroaryl-heteroaryl groups in which all of the parent heteroaromatic ring systems are identical.

“Biheteroaryl” by itself or as part of another substituent refers to a heteroaryl-heteroaryl group having two identical parent heteroaromatic ring systems joined directly together by a single bond. Typical biheteroaryl groups include, but are not limited to, bipyridyl, bipurinyl, biquinolinyl, and the like. Preferably, the heteroaromatic ring systems are 5-15 membered heteroaromatic rings, more preferably 5-10 membered heteroaromatic rings.

“Heteroarylalkyl” by itself or as part of another substituent refers to an acyclic alkyl group in which one of the hydrogen atoms bonded to a carbon atom, typically a terminal or sp³ carbon atom, is replaced with a heteroaryl group. Where specific alkyl moieties are intended, the nomenclature heteroarylalkanyl, heteroarylakenyl and/or heteroarylalkynyl is used. In preferred embodiments, the heteroarylalkyl group is a 6-21 membered heteroarylalkyl, e.g., the alkanyl, alkenyl or alkynyl moiety of the heteroarylalkyl is (C1-C6) alkyl and the heteroaryl moiety is a 5-15-membered heteroaryl. In particularly preferred embodiments, the heteroarylalkyl is a 6-13 membered heteroarylalkyl, e.g., the alkanyl, alkenyl or alkynyl moiety is (C1-C3) alkyl and the heteroaryl moiety is a 5-10 membered heteroaryl.

“Halogen” or “Halo” by themselves or as part of another substituent, unless otherwise stated, refer to fluoro, chloro, bromo and iodo.

“Haloalkyl” by itself or as part of another substituent refers to an alkyl group in which one or more of the hydrogen atoms is replaced with a halogen. Thus, the term “haloalkyl” is meant to include monohaloalkyls, dihaloalkyls, trihaloalkyls, etc. up to perhaloalkyls. For example, the expression “(C1-C2) haloalkyl” includes fluoromethyl, difluoromethyl, trifluoromethyl, 1-fluoroethyl, 1,1-difluoroethyl, 1,2-difluoroethyl, 1,1,1-trifluoroethyl, perfluoroethyl, etc.

The above-defined groups may include prefixes and/or suffixes that are commonly used in the art to create additional well-recognized substituent groups. As examples, “alkyloxy” or “alkoxy” refers to a group of the formula —OR″, “alkylamine” refers to a group of the formula-NHR″ and “dialkylamine” refers to a group of the formula-NR″R″, where each R″ is independently an alkyl. As another example, “haloalkoxy” or “haloalkyloxy” refers to a group of the formula —OR′″, where R′″ is a haloalkyl.

“Protecting group” refers to a group of atoms that, when attached to a reactive functional group in a molecule, mask, reduce or prevent the reactivity of the functional group. Typically, a protecting group may be selectively removed as desired during the course of a synthesis. Examples of protecting groups can be found in Greene and Wuts, Protective Groups in Organic Chemistry, 3^{rd} Ed., 1999, John Wiley & Sons, NY and Harrison et al., Compendium of Synthetic Organic Methods, Vols. 1-8, 1971-1996, John Wiley & Sons, NY. Representative amino protecting groups include, but are not limited to, formyl, acetyl, trifluoroacetyl, benzyl, benzyloxycarbonyl (“CBZ”), tert-butoxycarbonyl (“Boc”), trimethylsilyl (“TMS”), 2-trimethylsilyl-ethanesulfonyl (“TES”), trityl and substituted trityl groups, allyloxycarbonyl, 9-fluorenylmethyloxycarbonyl (“FMOC”), nitro-veratryloxycarbonyl (“NVOC”) and the like. Representative hydroxyl protecting groups include, but are not limited to, those where the hydroxyl group is either acylated or alkylated such as benzyl and trityl ethers, as well as alkyl ethers, tetrahydropyranyl ethers, trialkylsilyl ethers (e.g., TMS or TIPPS groups) and allyl ethers.

“Prodrug” refers to a derivative of an active JAK-inhibitory compound (drug) that requires a transformation under the conditions of use, such as within the body, to release the active drug. Prodrugs are frequently, but not necessarily, pharmacologically inactive until converted into the active drug. Prodrugs are typically obtained by masking a functional group in the drug believed to be in part required for activity with a progroup (defined below) to form a promoiety which undergoes a transformation, such as cleavage, under the specified conditions of use to release the functional group, and hence the active drug. The cleavage of the promoiety may proceed spontaneously, such as by way of a hydrolysis reaction, or it may be catalyzed or induced by another agent, such as by an enzyme, by light, by acid or base, or by a change of or exposure to a physical or environmental parameter, such as a change of temperature. The agent may be endogenous to the conditions of use, such as an enzyme present in the cells to which the prodrug is administered or the acidic conditions of the stomach, or it may be supplied exogenously.

A wide variety of progroups, as well as the resultant promoieties, suitable for masking functional groups in the active JAK selective inhibitory compounds to yield prodrugs are well-known in the art. For example, a hydroxyl functional group may be masked as a sulfonate, ester or carbonate promoiety, which may be hydrolyzed in vivo to provide the hydroxyl group. An amino functional group may be masked as an amide, carbamate, imine, urea, phosphenyl, phosphoryl or sulfenyl promoiety, which may be hydrolyzed in vivo to provide the amino group. A carboxyl group may be masked as an ester (including silyl esters and thioesters), amide or hydrazide promoiety, which may be hydrolyzed in vivo to provide the carboxyl group. Other specific examples of suitable progroups and their respective promoieties will be apparent to those of skill in the art.

“Progroup” refers to a type of protecting group that, when used to mask a functional group within an active drug to form a promoiety, converts the drug into a prodrug. Progroups are typically attached to the functional group of the drug via bonds that are cleavable under specified conditions of use. Thus, a progroup is that portion of a promoiety that cleaves to release the functional group under the specified conditions of use. As a specific example, an amide promoiety of the formula —NH—C(O)CH₃ comprises the progroup —C(O)CH₃.

5.2 The JAK Selective Inhibitory Compounds

As mentioned in the Summary, the JAK-selective compounds are generally 2,4-pyimidinediamines that bear specified substituent groups at the N2 nitrogen, the N4 nitrogen and optionally the 5-position of the pyrimidine ring. Specific exemplary JAK-selective inhibitory compounds are illustrated in TABLES 1 and 2. All of the compounds illustrated in TABLES 1 and 2 are selective for a JAK kinase as compared to Syk kinase, as determined by comparing the IC₅₀ obtained in a cellular mast cell degranulation assay with the IC₅₀ obtained in a cellular IL-4 activation asay (see the assays provided in the Examples section). The IC₅₀ ratios for each compound, and hence their “degree” of selectivity for a JAK kinase as compared to Syk kinase, are provided in the TABLES.

While the compounds illustrated in TABLE 2 are JAK selective, they also inhibit Syk kinase to some degree. For example, the IC₅₀ values in the mast cell degranulation assay range from approximately 0.1-20 μM, with most being in the 1-5 μM range. Thus, the compounds illustrated in TABLE 2 are essentially JAK-selective “dual” JAK/Syk inhibitors.

In stark contrast, the compounds illustrated in TABLE 1 do not appreciably inhibit Syk kinase. All of these compounds exhibit IC₅₀s in the mast cell degranulation assay that are at least about 50 μM. The selectivity ratios provided in TABLE 1 are the minimum ratio for the specified compounds, as they were calculated using an IC₅₀ of Syk inhibition of 50 μM which was the approximate upper limit of methodology used to carry out the assay.

All of the compounds in TABLES 1 and 2 are potent inhibitors of JAK kinases, exhibiting IC₅₀s in the IL-4 activation assay in the range of less than 5 μM, with most being in the nanomolar, and several in the sub-nanomolar, range.

Those of skill in the art will appreciate that the JAK-selective inhibitory compounds described herein may include functional groups that can be masked with progroups to create prodrugs. Such prodrugs are usually, but need not be, pharmacologically inactive until converted into their active drug form. Indeed, many of the JAK-selective inhibitory compounds described in TABLES 1 and 2 include promoieties that are hydrolyzable or otherwise cleavable under conditions of use. For example, ester groups commonly undergo acid-catalyzed hydrolysis to yield the parent carboxylic acid when exposed to the acidic conditions of the stomach, or base-catalyzed hydrolysis when exposed to the basic conditions of the intestine or blood. Thus, when administered to a subject orally, JAK-selective inhibitory compounds that include ester moieties may be considered prodrugs of their corresponding carboxylic acid, regardless of whether the ester form is pharmacologically active.

In the prodrugs, any available functional moiety may be masked with a progroup to yield a prodrug. Functional groups within the JAK-selective inhibitory compounds that may be masked with progroups for inclusion in a promoiety include, but are not limited to, amines (primary and secondary), hydroxyls, sulfanyls (thiols), carboxyls, etc. Myriad progroups suitable for masking such functional groups to yield promoieties that are cleavable under the desired conditions of use are known in the art. All of these progroups, alone or in combinations, may be included in the prodrugs.

Those of skill in the art will appreciate that many of the compounds and prodrugs described herein, as well as the various compound species specifically described and/or illustrated herein, may exhibit the phenomena of tautomerism, conformational isomerism, geometric isomerism and/or optical isomerism. For example, the compounds and prodrugs may include one or more chiral centers and/or double bonds and as a consequence may exist as stereoisomers, such as double-bond isomers (i.e., geometric isomers), enantiomers and diasteromers and mixtures thereof, such as racemic mixtures. As another example, the compounds and prodrugs may exist in several tautomeric forms, including the enol form, the keto form and mixtures thereof. As the various compound names, formulae and compound drawings within the specification and claims can represent only one of the possible tautomeric, conformational isomeric, optical isomeric or geometric isomeric forms, it should be understood that the invention encompasses any tautomeric, conformational isomeric, optical isomeric and/or geometric isomeric forms of the compounds or prodrugs having one or more of the utilities described herein, as well as mixtures of these various different isomeric forms. In cases of limited rotation around the 2,4-pryimidinediamine core structure, atrop isomers are also possible and are also specifically included in the compounds of the invention.

Moreover, skilled artisans will appreciate that when lists of alternative substituents include members which, owing to valency requirements or other reasons, cannot be used to substitute a particular group, the list is intended to be read in context to include those members of the list that are suitable for substituting the particular group. For example, skilled artisans will appreciate that while virtually any substitutent group can be used to substitute an alkyl group, certain of the substituent groups, such as ═O, cannot be used to substitute a phenyl group. It is to be understood that only possible combinations of substituent-group pairs are intended.

The compounds and/or prodrugs may be identified by either their chemical structure or their chemical name. When the chemical structure and the chemical name conflict, the chemical structure is determinative of the identity of the specific compound.

Depending upon the nature of the various substituents, the JAK-selective inhibitory compounds and prodrugs may be in the form of salts. Such salts include salts suitable for pharmaceutical uses (“pharmaceutically-acceptable salts”), salts suitable for veterinary uses, etc. Such salts may be derived from acids or bases, as is well-known in the art.

In one embodiment, the salt is a pharmaceutically acceptable salt. Generally, pharmaceutically acceptable salts are those salts that retain substantially one or more of the desired pharmacological activities of the parent compound and which are suitable for administration to humans. Pharmaceutically acceptable salts include acid addition salts formed with inorganic acids or organic acids. Inorganic acids suitable for forming pharmaceutically acceptable acid addition salts include, by way of example and not limitation, hydrohalide acids (e.g., hydrochloric acid, hydrobromic acid, hydriodic, etc.), sulfuric acid, nitric acid, phosphoric acid, and the like. Organic acids suitable for forming pharmaceutically acceptable acid addition salts include, by way of example and not limitation, acetic acid, trifluoroacetic acid, propionic acid, hexanoic acid, cyclopentanepropionic acid, glycolic acid, oxalic acid, pyruvic acid, lactic acid, malonic acid, succinic acid, malic acid, maleic acid, fumaric acid, tartaric acid, citric acid, palmitic acid, benzoic acid, 3-(4-hydroxybenzoyl) benzoic acid, cinnamic acid, mandelic acid, alkylsulfonic acids (e.g., methanesulfonic acid, ethanesulfonic acid, 1,2-ethane-disulfonic acid, 2-hydroxyethanesulfonic acid, etc.), arylsulfonic acids (e.g., benzenesulfonic acid, 4-chlorobenzenesulfonic acid, 2-naphthalenesulfonic acid, 4-toluenesulfonic acid, camphorsulfonic acid, etc.), 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like.

Pharmaceutically acceptable salts also include salts formed when an acidic proton present in the parent compound is either replaced by a metal ion (e.g., an alkali metal ion, an alkaline earth metal ion or an aluminum ion) or coordinates with an organic base (e.g., ethanol amine, diethanolamine, triethanolamine, N-methylglucamine, morpholine, piperidine, dimethylamine, diethylamine, etc.).

The JAK-selective inhibitory compounds and prodrugs described herein, as well as the salts thereof, may also be in the form of hydrates, solvates and N-oxides, as are well-known in the art.

5.3 Methods of Synthesis

The JAK selective inhibitory compounds and prodrugs may be synthesized via a variety of different synthetic routes using commercially available starting materials and/or starting materials prepared by conventional synthetic methods. Suitable exemplary methods are described in U.S. publication no. US 2004/0029902 (Ser. No. 10/355,543 filed Jan. 31, 2003) the disclosure of which is incorporated herein by reference.

5.4 Inhibition of JAK Kinases

The activity of a specified compound as an inhibitor of a JAK kinase may be assessed in vitro or in vivo. In some embodiments, the activity of a specified compound can be tested in a cellular assay. Suitable assays include assays that determine inhibition of either the phosphorylation activity or ATPase activity of an activated JAK kinase. Thus, a compound is said to inhibit an activity of a JAK kinase if it inhibits the phosphorylation or ATPase activity of an activated JAK kinase with an IC₅₀ of about 10 μM or less. A specific assay for assessing JAK kinase inhibition, and in particular JAK1 and/or JAK3 kinase inhibition, is described in the Examples section.

Similar types of assays can be used to assess Syk kinase inhibitory activity to determine the degree of selectivity of the particular compound as compared to Syk kinase. Suitable assays for assessing Syk kinase inhibition, for example assays that assess inhibition of IgE mediated mast cell degranulation, are described in U.S. publication no. US 2004/0029902 (Ser. No. 10/355,543 filed Jan. 30, 2003), the disclosure of which is incorporated by reference. Selectivity could also be ascertained in biochemical assays with isolated kinases.

5.5 Uses and Compositions

As previously discussed, the compounds described herein are potent and selective inhibitors of JAK kinases. As a consequence of this activity, the compounds may be used in a variety of in vitro, in vivo and ex vivo contexts to regulate or inhibit JAK kinase activity, signaling cascades in which JAK kinases play a role, and the biological responses effected by such signaling cascades. For example, in one embodiment, the compounds may be used to inhibit JAK kinase, either in vitro or in vivo, in virtually any cell type expressing the JAK kinase. They may also be used to regulate signal transduction cascades in which JAK kinases play a role. Such JAK-dependent signal transduction cascades include, but are not limited to, the signaling cascades of cytokine receptors that involve the common gamma chain, such as, for example, the IL-3, IL-4, IL-7, IL-5, IL-9, IL-15 and IL-21 receptor signaling cascades. The compounds may also be used in vitro or in vivo to regulate, and in particular inhibit, cellular or biological responses effected by such JAK-dependent signal transduction cascades. Such cellular or biological responses include, but are not limited to, IL-4/ramos CD23 upregulation, IL-2 mediated T-cell proliferation, etc. Importantly, the compounds may be used to inhibit JAK kinases in vivo as a therapeutic approach towards the treatment or prevention of diseases mediated, either wholly or in part, by a JAK kinase activity (referred to herein as “JAK kinase mediated diseases”). Non-limiting examples of JAK kinase mediated diseases that may be treated or prevented with the compounds, include, but are not limited to, allergies, asthma, autoimmune diseases include; but are not limited to allergies, asthma, autoimmune diseases such as transplant rejection (e.g., kidney, heart, lung, liver, pancreas, skin; host versus graft reaction (HVGR), etc.), rheumatoid arthritis, and amyotrophic lateral sclerosis, T-cell mediated autoimmune diseases such as multiple sclerosis, psoraiasis and Sjogren's syndrome, Type II inflammatory diseases such as vascular inflammation (including vasculitis, arteritis, atherosclerosis and coronary artery disease), diseases of the central nervous system such as stroke, pulmonary diseases such as bronchitis obliteraus and primary pulmonary hypertension, and solid, delayed Type IV hypersensitivity reactions, and hematologic malignancies such as leukemia and lymphomas.

In a specific embodiment, the compounds can be used to treat and/or prevent rejection in organ and/or tissue transplant recipients (i.e., treat and/or prevent allorgraft rejection).

Allorafts may be rejected through either a cell-mediated or humoral immune reaction of the recipient against transplant (histocompability) antigens present on the membranes of the donor's cells. The strongest antigens are governed by a complex of genetic loci termed human leukocyte group A (HLA) antigens. Together with the ABO blood groups antigens, they are the chief transplantation antigens detectable in humans.

Rejection following transplantation can generally be broken into three categories: hyperacute, occurring hours to days following transplantation; acute, occurring days to months following transplantation; and chronic, occurring months to years following transplantation.

Hyperacute rejection is caused mainly by the production of host antibodies that attack the graft tissue. In a hyperacute rejection reaction, antibodies are observed in the transplant vascular very soon after transplantation. Shortly thereafter, vascular clotting occurs, leading to ischemia, eventual necrosis and death. The graft infarction is unresponsive to known immunosuppressive therapies. Because HLA antigens can be identified in vitro, pre-transplant screening is used to significantly reduce hyperacute rejection. As a consequence of this screening, hyperacute rejection is relative uncommon today.

Acute rejection is thought to be mediated by the accumulation of antigen specific cells in the graft tissue. The T-cell-mediated immune reaction against these antigens (i.e., the HVGR) is the principle mechanism of acute rejection. Accumulation of these cells leads to damage of the graft tissue through, for example, the host CTL, innocent by standard reactions and/or vascular thrombosis. It is believed that both CD4+ helper T-cells and CD8+ cytotoxic T-cells are involved in the process, and that the antigen is presented by donor and host dendritic cells. The CD4+ helper T-cells help recruit other effector cells, such as macrophapges and cosinophils, to the graft. Accessing T-cell activation signal transduction cascades (for example, CD28, CD40L and CD2 cascades) are also involved.

Although the graft tissue can suffer from varying degrees of hemorrhage and edema, the vascular integrity is usually maintained, although the arterial endothelium appears to be a primary target of HVGR acute rejection.

The cell-mediated acute rejection may be reversed in many cases by intensifying immunotherapy. After successful reversal, severely damaged elements of the graft heal by fibrosis and the remainder of the graft appears normal. After resolution of acute rejection, dosages of immunosuppressive drugs can be reduced to very low levels.

Chronic rejection, which is a particular problem in renal transplants, often progresses insidiously despite increased immunosuppressive therapy. It is thought to be due, in large part, to cell-mediated Type IV hypersensitivity. The pathologic profile differs from that of acute rejection. The arterial andothelium is primarily involved, with extensive proliferation that may gradually occlude the vessel lumen, leading to ischemia, fibrosis, a thickened intima and atherosclerotic changes. Chronic rejection is mainly due to a progressive obliteration of graft vasculature, and resembles a slow, vasculitic process.

In Type IV hypersensitivity, CD8 cytotoxic T-cells and CD24 helper T cells recognize either intracellular as extracellular synthesized antigen when it is complexed, respectively, with either Class I, class II MHC molecular. Macrophages function as antigen-presenting cells and release IL-1, which promotes proliferation of helper T-cells. Helper T-cells release interferon gamma and IL-2, which together regulate delayed hyperactivity reactions mediated by macrophage activation and immunity mediated by T cells. In the case of organ transplant, the cytotoxic T-cells destroy the graft cells on contact.

Since JAK kinases play a critical role in the activation of T-cells, the JAK-selective inhibitory compounds described herein can be used to treat and/or prevent many aspects of transplant rejection, and are particularly useful in the treatment and/or prevention of rejection reactions that are mediated, at least in part, by T-cells, such as the HVGR. The JAK-selective compounds can also be used to treat and/or prevent chronic rejection in transplant recipients, and in particular in renal transplant recipients.

The JAK-selective therapy can be applied alone, or it can be applied in combination with or adjunctive to other common immunosuppressive therapies, such as, for example, mercaptopurine, cortisosteroids such as prednisone, methylprednisolone and prednisolone, alkylating agents such as cyclophosphamide, calcineurin inhibitors such as cyclosporine, sirolimus and tacrolimus, inhibitors of inosine monophosphate dehydrogenase (IMPDH) such as mycophenolate, mycophenolate mofetil and azathioprine, and agents designed to suppress cellular immunity while leaving the recipient's humoral immunologic response intact, including various antibodies (for example, antilymphocyte globulin (ALG), antithymocyte globulin (ATG), monoclonal anti-T-cell antibodies (OKT3)) and irradiation. These various agents can be used in accordance with their standard or common dosages, as specified in the prescribing information accompanying commercially available forms of the drugs (see also, the prescribing information in the 2005 Edition of The Physician's Desk Reference), the disclosures of which are incorporated herein by reference. Azothiopurine is current available from Salix Pharmaceuticals, Inc. under the brand name AZASAN; mercaptopurine is currently available from Gate Pharmaceuticals, Inc. under the brand name PURINETHOL; prednisone and prednisolone are currently available from Roxane Laboratories, Inc.; Methyl prednisolone is currently available from Pfizer; sirolimus (rapamycin) is currently available from Wyeth-Ayerst under the brand name RAPAMUNE; tacrolimus is currently available from Fujisawa under the brand name PROGRAF; cyclosporine is current available from Novartis under the brand dame SANDIMMUNE and Abbott under the brand name GENGRAF; IMPDH inhibitors such as mycophenolate mofetil and mycophenolic acid are currently available from Roche under the brand name CELLCEPT and Novartis under the brand name MYFORTIC; azathioprine is currently available from Glaxo Smith Kline under the brand name IMURAN; and antibodies are currently available from Ortho Biotech under the brand name ORTHOCLONE, Novartis under the brand name SIMULECT (basiliximab) and Roche under the brand name ZENAPAX (daclizumab).

In a specific embodiment, the JAK-selective compounds could be administered either in combination or adjunctively with a Syk inhibitory compound. Suitable Syk inhibitory compounds are described, for example, in Ser. No. 10/355,543 filed Jan. 31, 2003 (publication no. 2004/0029902); WO 03/063794; Ser. No. 10/631,029 filed Jul. 29, 2003; WO 2004/014382; Ser. No. 10/903,263 filed Jul. 30, 2004; PCT/US2004/24716 filed Jul. 30, 2004; Ser. No. 10/903,870 filed Jul. 30, 2004; PCT/US2004/24920 filed Jul. 30, 2004; Ser. No. 60/630,808 filed Nov. 24, 2004; Ser. No. 60/645,424 filed Jan. 19, 2005; and Ser. No. 60/654,620 filed Feb. 18, 2005, the disclosures of which are incorporated herein by reference. The JAK-selective and Syk inhibitory compounds could be used alone, or in combination with one or more conventional transplant rejection treatments, as described above.

In a specific embodiment, the JAK-selective compounds can be used to treat or prevent these diseases in patients that are either initially non-responsive to (resistant), or that become non-responsive to, treatment with a Syk inhibitory compound, or one of the other current treatments for the particular disease. The JAK-selective compounds could also be used in combination with Syk inhibitory compounds in patients that are Syk-compound resistant or non-responsive. Suitable Syk-inhibitory compounds with which the JAK-selective compounds can be administered are provided supra.

The JAK-selective inhibitory compounds described herein are cytokine modulators of IL-4 and IL-13 signaling. As a consequence, the JAK-selective compounds could slow the response of Type I hypersensitivity reactions. Thus, in a specific embodiment, the JAK-selective compounds could be used to treat such reactions, and therefore the diseases associated with, mediated by or caused by such hypersensitivity reactions (for example, allergies), prophylactically. For example, an allergy sufferer could take one or more of the JAK selective compounds described herein prior to expected exposure to allergens to delay the onset or progress, or eliminate altogether, an allergic response.

When used to treat or prevent such diseases, the JAK-selective compounds may be administered singly, as mixtures of one or more JAK-selective compounds or in mixture or combination with other agents useful for treating such diseases and/or the symptoms associated with such diseases. The JAK-selective compounds may also be administered in mixture or in combination with agents useful to treat other disorders or maladies, such as steroids, membrane stabilizers, 5LO inhibitors, leukotriene synthesis and receptor inhibitors, inhibitors of IgE isotype switching or IgE synthesis, IgG isotype switching or IgG synthesis, β-agonists, tryptase inhibitors, aspirin, COX inhibitors, methotrexate, anti-TNF drugs, retuxin, PD4 inhibitors, p38 inhibitors, PDE4 inhibitors, and antihistamines, to name a few. The JAK-selective compounds may be administered per se in the form of prodrugs or as pharmaceutical compositions, comprising an active compound or prodrug.

Pharmaceutical compositions comprising the JAK-selective compounds described herein (or prodrugs thereof) may be manufactured by means of conventional mixing, dissolving, granulating, dragee-making levigating, emulsifying, encapsulating, entrapping or lyophilization processes. The compositions may be formulated in conventional manner using one or more physiologically acceptable carriers, diluents, excipients or auxiliaries which facilitate processing of the active compounds into preparations which can be used pharmaceutically.

The JAK-selective compound or prodrug may be formulated in the pharmaceutical compositions per se, or in the form of a hydrate, solvate, N-oxide or pharmaceutically acceptable salt, as previously described. Typically, such salts are more soluble in aqueous solutions than the corresponding free acids and bases, but salts having lower solubility than the corresponding free acids and bases may also be formed.

Pharmaceutical compositions of the invention may take a form suitable for virtually any mode of administration, including, for example, topical, ocular, oral, buccal, systemic, nasal, injection, transdermal, rectal, vaginal, etc., or a form suitable for administration by inhalation or insufflation.

For topical administration, the JAK-selective compound(s) or prodrug(s) may be formulated as solutions, gels, ointments, creams, suspensions, etc. as are well-known in the art.

Systemic formulations include those designed for administration by injection, e.g., subcutaneous, intravenous, intramuscular, intrathecal or intraperitoneal injection, as well as those designed for transdermal, transmucosal oral or pulmonary administration.

Useful injectable preparations include sterile suspensions, solutions or emulsions of the active compound(s) in aqueous or oily vehicles. The compositions may also contain formulating agents, such as suspending, stabilizing and/or dispersing agent. The formulations for injection may be presented in unit dosage form, e.g., in ampules or in multidose containers, and may contain added preservatives.

Alternatively, the injectable formulation may be provided in powder form for reconstitution with a suitable vehicle, including but not limited to sterile pyrogen free water, buffer, dextrose solution, etc., before use. To this end, the active compound(s) may be dried by any art-known technique, such as lyophilization, and reconstituted prior to use.

For transmucosal administration, penetrants appropriate to the barrier to be permeated are used in the formulation. Such penetrants are known in the art.

For oral administration, the pharmaceutical compositions may take the form of, for example, lozenges, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulfate). The tablets may be coated by methods well known in the art with, for example, sugars, films or enteric coatings.

Liquid preparations for oral administration may take the form of, for example, elixirs, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol, cremophore™ or fractionated vegetable oils); and preservatives (e.g., methyl or propyl-p-hydroxybenzoates or sorbic acid). The preparations may also contain buffer salts, preservatives, flavoring, coloring and sweetening agents as appropriate.

Preparations for oral administration may be suitably formulated to give controlled release of the active compound or prodrug, as is well known.

For buccal administration, the compositions may take the form of tablets or lozenges formulated in conventional manner.

For rectal and vaginal routes of administration, the active compound(s) may be formulated as solutions (for retention enemas) suppositories or ointments containing conventional suppository bases such as cocoa butter or other glycerides.

For nasal administration or administration by inhalation or insufflation, the active compound(s) or prodrug(s) can be conveniently delivered in the form of an aerosol spray from pressurized packs or a nebulizer with the use of a suitable propellant, e.g., dichlorodifluoromethane, trichlorofluoromethane, dichlorotetrafluoroethane, fluorocarbons, carbon dioxide or other suitable gas. In the case of a pressurized aerosol, the dosage unit may be determined by providing a valve to deliver a metered amount. Capsules and cartridges for use in an inhaler or insufflator (for example capsules and cartridges comprised of gelatin) may be formulated containing a powder mix of the compound and a suitable powder base such as lactose or starch.

For ocular administration, the JAK-selective compound(s) or prodrug(s) may be formulated as a solution, emulsion, suspension, etc. suitable for administration to the eye. A variety of vehicles suitable for administering compounds to the eye are known in the art. Specific non-limiting examples are described in U.S. Pat. No. 6,261,547; U.S. Pat. No. 6,197,934; U.S. Pat. No. 6,056,950; U.S. Pat. No. 5,800,807; U.S. Pat. No. 5,776,445; U.S. Pat. No. 5,698,219; U.S. Pat. No. 5,521,222; U.S. Pat. No. 5,403,841; U.S. Pat. No. 5,077,033; U.S. Pat. No. 4,882,150; and U.S. Pat. No. 4,738,851.

For prolonged delivery, the JAK-selective compound(s) or prodrug(s) can be formulated as a depot preparation for administration by implantation or intramuscular injection. The active ingredient may be formulated with suitable polymeric or hydrophobic materials (e.g., as an emulsion in an acceptable oil) or ion exchange resins, or as sparingly soluble derivatives, e.g., as a sparingly soluble salt. Alternatively, transdermal delivery systems manufactured as an adhesive disc or patch which slowly releases the active compound(s) for percutaneous absorption may be used. To this end, permeation enhancers may be used to facilitate transdermal penetration of the active compound(s). Suitable transdermal patches are described in for example, U.S. Pat. No. 5,407,713.; U.S. Pat. No. 5,352,456; U.S. Pat. No. 5,332,213; U.S. Pat. No. 5,336,168; U.S. Pat. No. 5,290,561; U.S. Pat. No. 5,254,346; U.S. Pat. No. 5,164,189; U.S. Pat. No. 5,163,899; U.S. Pat. No. 5,088,977; U.S. Pat. No. 5,087,240; U.S. Pat. No. 5,008,110; and U.S. Pat. No. 4,921,475.

Alternatively, other pharmaceutical delivery systems may be employed. Liposomes and emulsions are well-known examples of delivery vehicles that may be used to deliver active compound(s) or prodrug(s). Certain organic solvents such as dimethylsulfoxide (DMSO) may also be employed, although usually at the cost of greater toxicity.

The pharmaceutical compositions may, if desired, be presented in a pack or dispenser device which may contain one or more unit dosage forms containing the active compound(s). The pack may, for example, comprise metal or plastic foil, such as a blister pack. The pack or dispenser device may be accompanied by instructions for administration.

5.6 Effective Dosages

The JAK-selective compound(s) or prodrug(s) described herein, or compositions thereof, will generally be used in an amount effective to achieve the intended result, for example in an amount effective to treat or prevent the particular disease being treated. The compound(s) may be administered therapeutically to achieve therapeutic benefit or prophylactically to achieve prophylactic benefit. By therapeutic benefit is meant eradication or amelioration of the underlying disorder being treated and/or eradication or amelioration of one or more of the symptoms associated with the underlying disorder such that the patient reports an improvement in feeling or condition, notwithstanding that the patient may still be afflicted with the underlying disorder. For example, administration of a compound to a patient suffering from an allergy provides therapeutic benefit not only when the underlying allergic response is eradicated or ameliorated, but also when the patient reports a decrease in the severity or duration of the symptoms associated with the allergy following exposure to the allergen. As another example, therapeutic benefit in the context of asthma includes an improvement in respiration following the onset of an asthmatic attack, or a reduction in the frequency or severity of asthmatic episodes. As another specific example, therapeutic benefit in the context of transplantation rejection includes the ability to alleviate an acute rejection episode, such as for example, the HVGR, or the ability to prolong the time period between onset of acute rejection episodes and/or onset of chronic rejection. Therapeutic benefit also includes halting or slowing the progression of the disease, regardless of whether improvement is realized.

For prophylactic administration, the compound may be administered to a patient at risk of developing one of the previously described diseases. For example, if it is unknown whether a patient is allergic to a particular drug, the compound may be administered prior to administration of the drug to avoid or ameliorate an allergic response to the drug. Alternatively, prophylactic administration may be applied to avoid the onset of symptoms in a patient diagnosed with the underlying disorder. For example, a compound may be administered to an allergy sufferer prior to expected exposure to the allergen. Compounds may also be administered prophylactically to healthy individuals who are repeatedly exposed to agents known to one of the above-described maladies to prevent the onset of the disorder. For example, a compound may be administered to a healthy individual who is repeatedly exposed to an allergen known to induce allergies, such as latex, in an effort to prevent the individual from developing an allergy. Alternatively, a compound may be administered to a patient suffering from asthma prior to partaking in activities which trigger asthma attacks to lessen the severity of, or avoid altogether, an asthmatic episode.

In the context of transplant rejection, the compound may be administered while the patient is not having an acute rejection reaction to avoid the onset of rejection and/or prior to the appearance of clinical indications of chronic rejection.

The amount of compound administered will depend upon a variety of factors, including, for example, the particular indication being treated, the mode of administration, whether the desired benefit is prophylactic or therapeutic, the severity of the indication being treated and the age and weight of the patient, the bioavailability of the particular active compound, etc. Determination of an effective dosage is well within the capabilities of those skilled in the art.

Effective dosages may be estimated initially from in vitro assays. For example, an initial dosage for use in animals may be formulated to achieve a circulating blood or serum concentration of active compound that is at or above an IC₅₀ of the particular compound as measured in as in vitro assay. Calculating dosages to achieve such circulating blood or serum concentrations taking into account the bioavailability of the particular compound is well within the capabilities of skilled artisans. For guidance, the reader is referred to Fingl & Woodbury, “General Principles,” In: Goodman and Gilman's The Pharmaceutical Basis of Therapeutics, Chapter 1, pp. 1-46, latest edition, Pagamonon Press, and the references cited therein.

Initial dosages can also be estimated from in vivo data, such as animal models. Animal models useful for testing the efficacy of compounds to treat or prevent the various diseases described above are well-known in the art. Suitable animal models of hypersensitivity or allergic reactions are described in Foster, 1995, Allergy 50(21Suppl):6-9, discussion 34-38 and Tumas et al., 2001, J. Allergy Clin. Immunol. 107(6):1025-1033. Suitable animal models of allergic rhinitis are described in Szelenyi et al., 2000, Arzneimittelforschung 50(11):1037-42; Kawaguchi et al., 1994, Clin. Exp. Allergy 24(3):238-244 and Sugimoto et al., 2000, Immunopharmacology 48(1):1-7. Suitable animal models of allergic conjunctivitis are described in Carreras et al., 1993, Br. J. Ophthalmol. 77(8):509-514; Saiga et al., 1992, Ophthalmic Res. 24(1):45-50; and Kunert et al., 2001, Invest. Ophthalmol. Vis. Sci. 42(11):2483-2489. Suitable animal models of systemic mastocytosis are described in O'Keefe et al., 1987, J. Vet. Intern. Med. 1(2):75-80 and Bean-Knudsen et al, 1989, Vet. Pathol. 26(1):90-92. Suitable animal models of hyper IgE syndrome are described in Claman et al., 1990, Clin. Immunol. Immunopathol. 56(1):46-53. Suitable animal models of B-cell lymphoma are described in Hough et al., 1998, Proc. Natl. Acad. Sci. USA 95:13853-13858 and Hakim et al., 1996, J. Immunol. 157(12):5503-5511. Suitable animal models of atopic disorders such as atopic dermatitis, atopic eczema and atopic asthma are described in Chan et al, 2001, J. Invest. Dermatol. 117(4):977-983 and Suto et al., 1999, Int. Arch. Allergy Immunol. 120(Suppl 1):70-75. Suitable animal models of transplant rejection, such as models of the HVGR are described in O'Shea et al., 2004, Nature Reviews Drug Discovery 3:555-564; Cetkovic-Curlje & Tibbles, 2004, Current Pharmaceutical Design 10:1767-1784; and Chengelian et al., 2003, Science 302:875-878. Ordinarily skilled artisans can routinely adapt such information to determine dosages suitable for human administration. Additional suitable animal models are described in the Examples section.

Dosage amounts will typically be in the range of from about 0.0001 or 0.001 or 0.01 mg/kg/day to about 100 mg/kg/day, but may be higher or lower, depending upon, among other factors, the activity of the compound, its bioavailability, the mode of administration and various factors discussed above. Dosage amount and interval may be adjusted individually to provide plasma levels of the compound(s) which are sufficient to maintain therapeutic or prophylactic effect. For example, the compounds may be administered once per week, several times per week (e.g., every other day), once per day or multiple times per day, depending upon, among other things, the mode of administration, the specific indication being treated and the judgment of the prescribing physician. In cases of local administration or selective uptake, such as local topical administration, the effective local concentration of active compound(s) may not be related to plasma concentration. Skilled artisans will be able to optimize effective local dosages without undue experimentation.

Preferably, the compound(s) will provide therapeutic or prophylactic benefit without causing substantial toxicity. Toxicity of the compound(s) may be determined using standard pharmaceutical procedures. The dose ratio between toxic and therapeutic (or prophylactic) effect is the therapeutic index. Compounds(s) that exhibit high therapeutic indices are preferred.

The various inventions having been described, the following examples are offered by way of illustration and not limitation.

### 6. EXAMPLES

6.1 The 2,4-Pyrimidinediamine Compounds Are Selective for JAK Kinasesas Compared to Syk

The selectivity of the compounds illustrated in TABLES 1 and 2 for JAK kinases as compared to Syk kinase was confirmed in cellular assays designed to test for JAK and Syk inhibition. Briefly, JAK inhibition was tested in human Ramos B-cells activated with IL-4. Twenty to 24 hours post stimulation, the cells are stained for upregulation of CD23 and analyzed by FACS. Stimulation of the B-cells with IL-4 leads to the activation of the JAK/STAT pathway through phosphorylation of the JAK kinase JAK1 and JAK3, which in turn phosphorylate and activate transcription of factor STAT-5. The low-affinity IgE receptor (CD23) is upregulated by activated STAT-5.

For the assay, human Ramos B-cells (ATCC, Catalog No. CRL-1596) are cultured in RPMI 1640 medium (Cellgro, Catalog No. 10-040-CM) containing 10% fetal bovine serum (JRH, Catalog No. 12106-500M) according to the propagation protocol supplied with the cells, and maintained at a density of approximately 3.5×10⁵ cells/ml. The day before the assay, the cells are diluted to 3.5×10⁵ cells/ml to insure they are in the logorithmic growth phase. The cells are spun down, and suspended in RPMI 1640 medium containing 5% fetal bovine serum to a density of 3.5×10⁴ cells/ml and aliquots dispensed into a 96-well tissue culture plate. Cells are incubated with test compound (dissolved in DMSO) or DMSO (control) for 1 hr at 37° C. and then stimulated with IL-4 (Pepotech, Catalog No. 200-04) for 20-24 hours (final concentration is 50 Units/ml). Cells are then spun down, stained with anti-CD23-PE antibody (BD Pharmigen, Catalog No.555711) and analyzed by FACS.

Syk inhibition was tested in CHMC cells activated with IgE antibodies as described in U.S. Ser. No. 10/355,543 filed Jan. 31, 2003 (publication no. 2004-0029902), the disclosure of which is incorporated herein by reference. For the Syk assay, the amount of tryptase released upon degranulation was measured. The degree of selectivity for each compound, reported as the ratio of the IC₅₀ (Syk)/IC₅₀ (JAK), is provided in TABLES 1 and 2. For the compounds listed in TABLE 1, the ratio was calculated using an IC₅₀ value of Syk inhibition of 50 μM, which was the appropriate upper limit of the assay.

TABLE 1

| | IC₅₀ Ratio | |
|---|---|---|
| Cmpd No. | (Syk/JAK) | Structure |
| 1 | 207 | 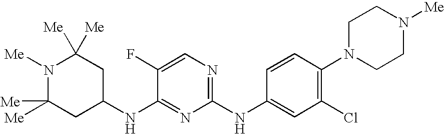 |
| 2 | 155 | 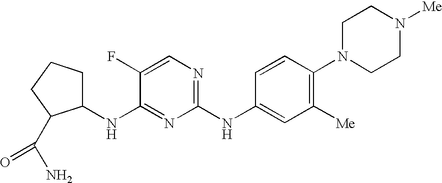 |
| 3 | 147 | 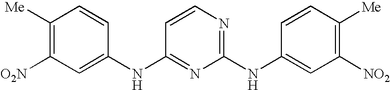 |
| 4 | 145 | 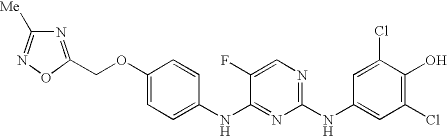 |
| 5 | 105 | 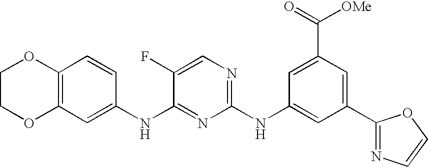 |
| 6 | 70 | 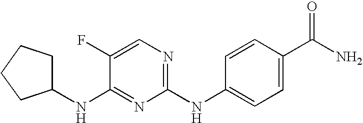 |
| 7 | 57 | 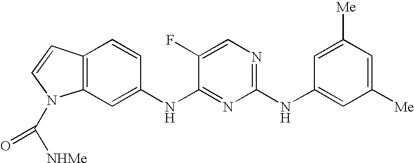 |
| 8 | 51 | 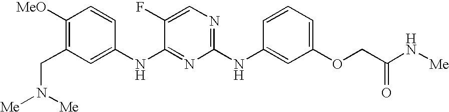 |
| 9 | 47 | 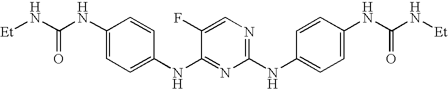 |
| 10 | 43 | 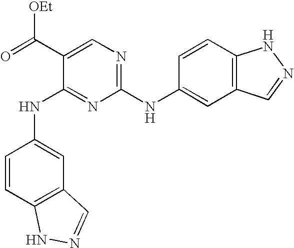 |
| 11 | 38 | 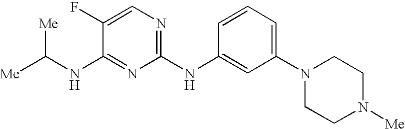 |
| 12 | 36 | 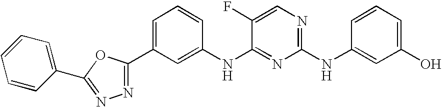 |
| 13 | 26 | 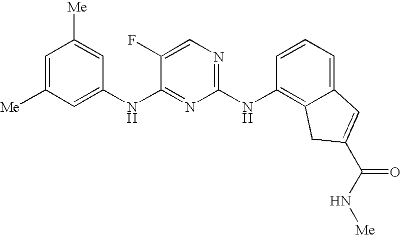 |
| 14 | 25 | 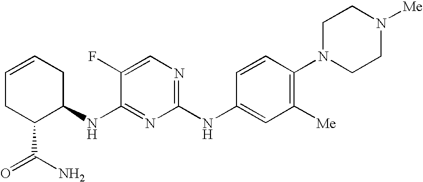 |
| 15 | 24 | 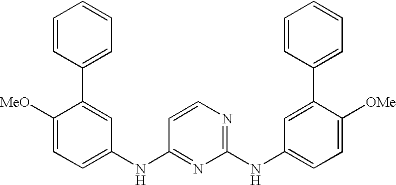 |
| 16 | 22 | 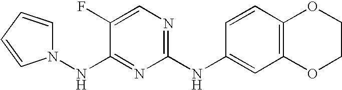 |
| 17 | 20 | 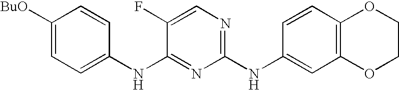 |
| 18 | 20 | 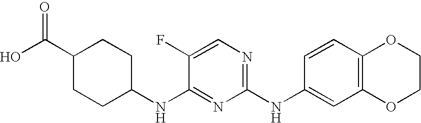 |
| 19 | 15 | 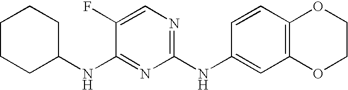 |
| 20 | 14 | 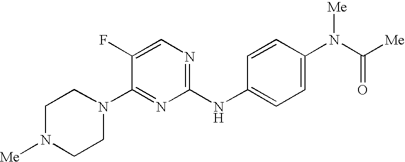 |
| 21 | 14 | 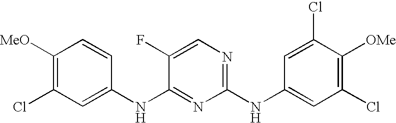 |
| 22 | 13 | 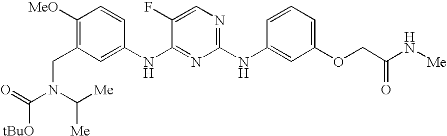 |
| 23 | 12 | 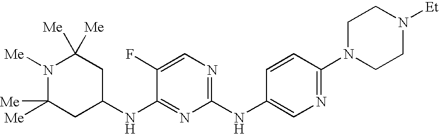 |
| 24 | 12 | 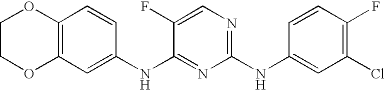 |
| 25 | 13 | 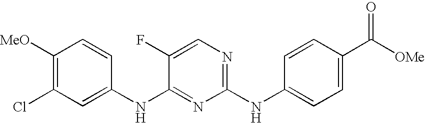 |
| 26 | 10 | 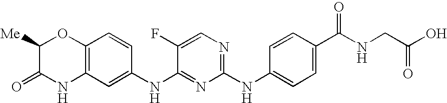 |

TABLE 2

| | IC₅₀ Ratio | |
|---|---|---|
| Cmpd No. | (Syk/JAK) | Structure |
| 27 | 247 | 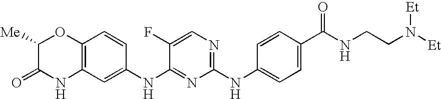 |
| 28 | 146 | 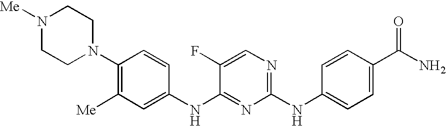 |
| 29 | 109 | 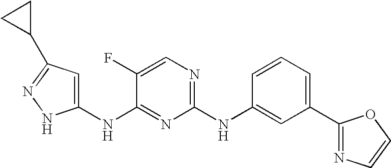 |
| 30 | 85 | 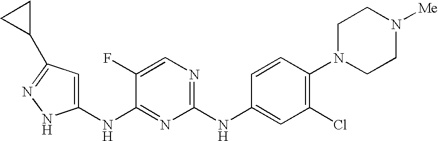 |
| 31 | 74 | 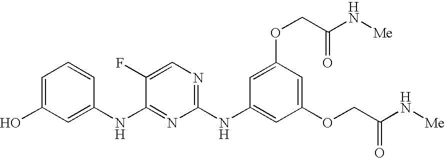 |
| 32 | 65 | 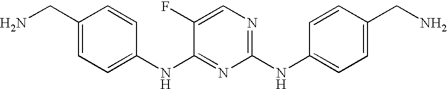 |
| 33 | 57 | 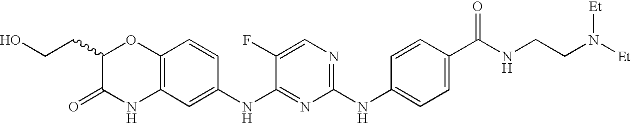 |
| 34 | 55 | 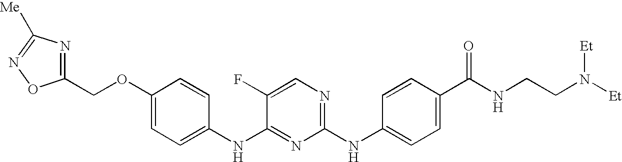 |
| 35 | 44 | 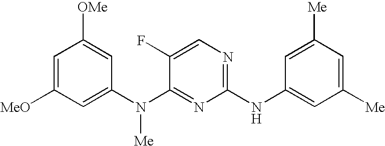 |
| 36 | 43 | 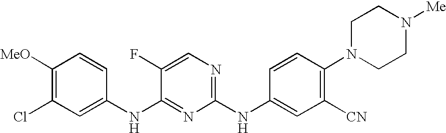 |
| 37 | 42 | 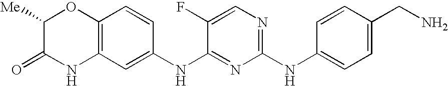 |
| 38 | 39 | 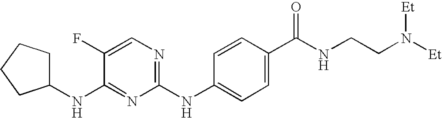 |
| 39 | 59 | 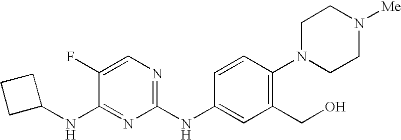 |
| 40 | 32 | 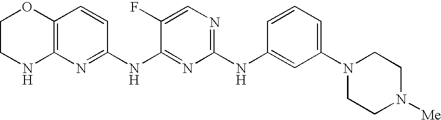 |
| 41 | 32 | 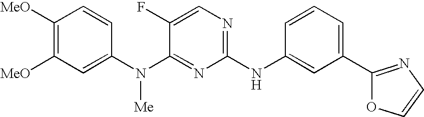 |
| 42 | 32 | 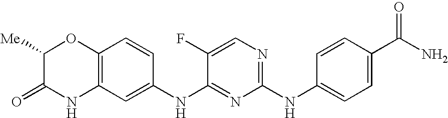 |
| 43 | 31 | 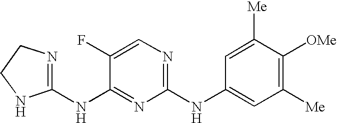 |
| 44 | 30 | 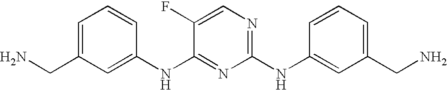 |
| 45 | 30 | 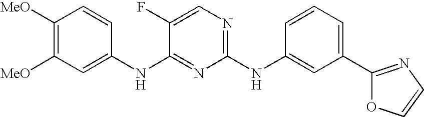 |
| 46 | 30 | 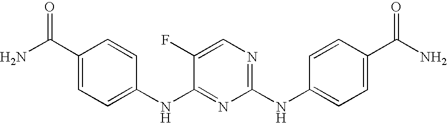 |
| 47 | 29 | 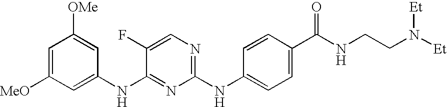 |
| 48 | 28 | 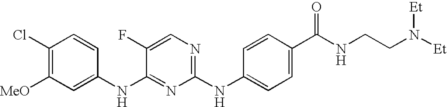 |
| 49 | 26 | 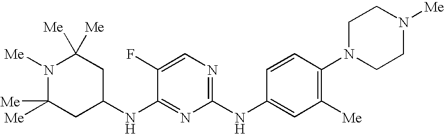 |
| 50 | 26 | 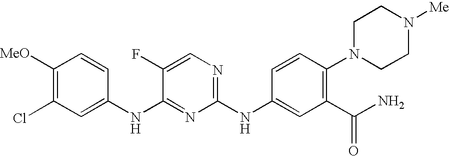 |
| 51 | 25 | 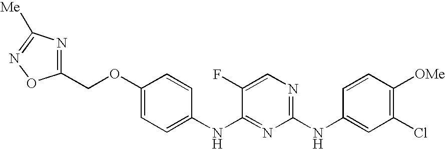 |
| 52 | 25 | 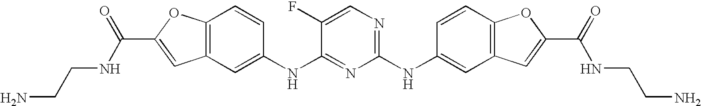 |
| 53 | 24 | 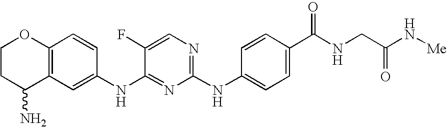 |
| 54 | 24 | 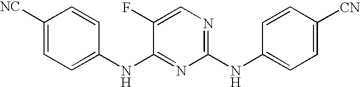 |
| 55 | 23 | 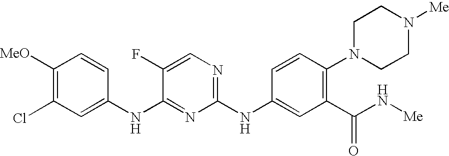 |
| 56 | 21 | 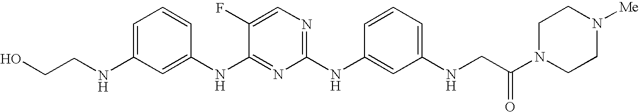 |
| 57 | 21 | 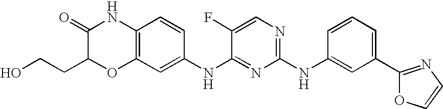 |
| 58 | 21 | 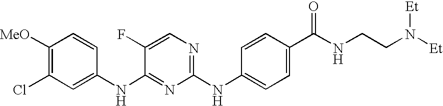 |
| 59 | 21 | 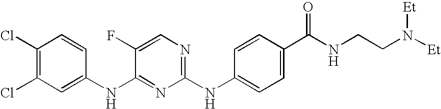 |
| 60 | 20 | 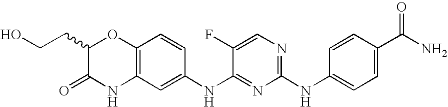 |
| 61 | 20 | 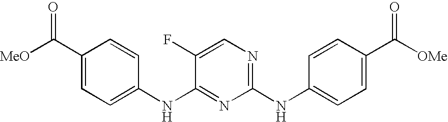 |
| 62 | 19 | 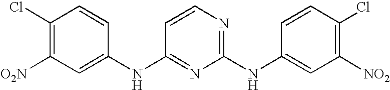 |
| 63 | 19 | 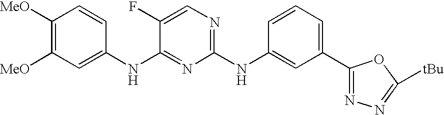 |
| 64 | 19 | 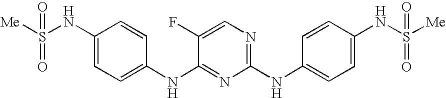 |
| 65 | 18 | 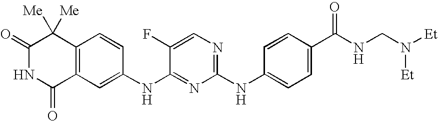 |
| 66 | 17 | 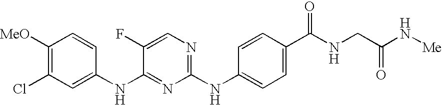 |
| 67 | 17 | 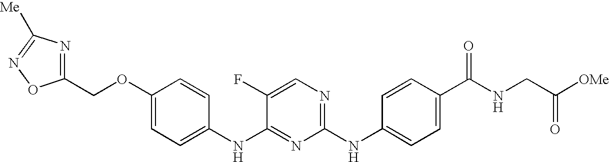 |
| 68 | 16 | 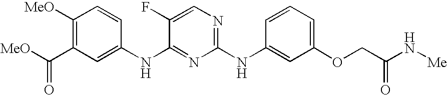 |
| 69 | 16 | 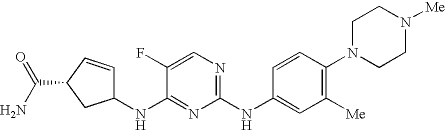 |
| 70 | 15 | 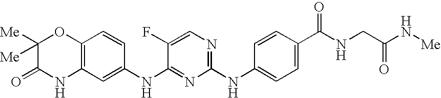 |
| 71 | 15 | 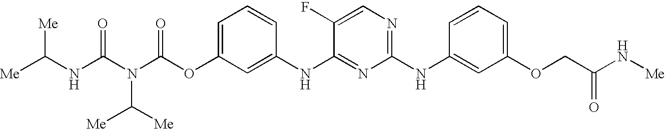 |
| 72 | 14 | 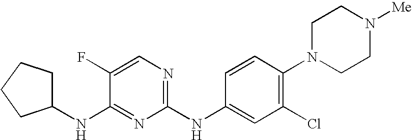 |
| 73 | 14 | 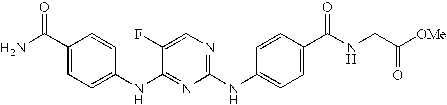 |
| 74 | 14 | 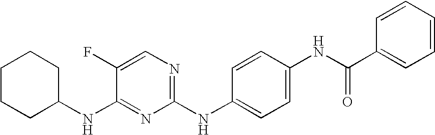 |
| 75 | 14 | 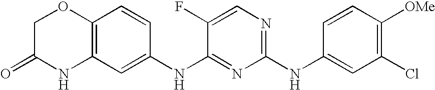 |
| 76 | 14 | 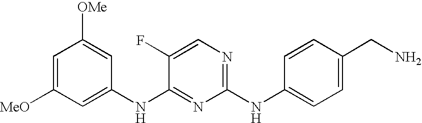 |
| 77 | 13 | 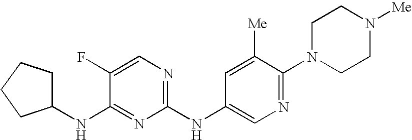 |
| 78 | 13 | 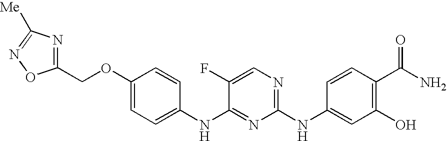 |
| 79 | 12 | 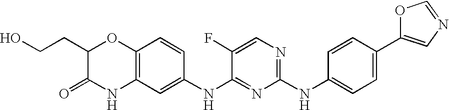 |
| 80 | 11 | 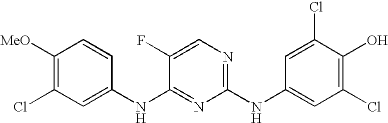 |
| 81 | 11 | 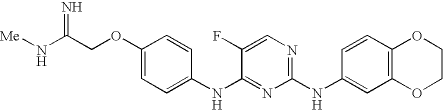 |
| 82 | 10 | 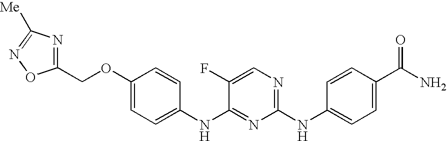 |
| 83 | 10 | 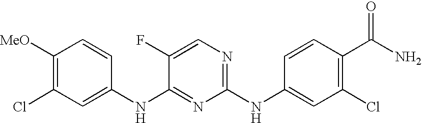 |
| 84 | 10 | 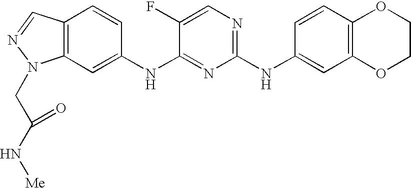 |
| 85 | 10 | 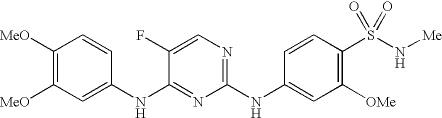 |
| 86 | 10 | 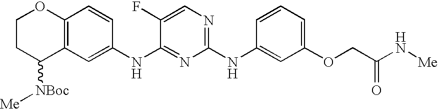 |
| 87 | 10 | 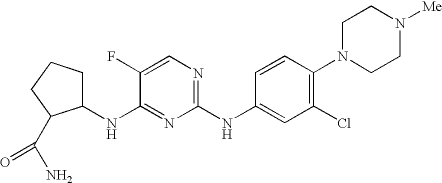 |
| 88 | 10 | 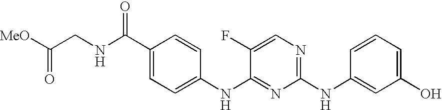 |
| 89 | 10 | 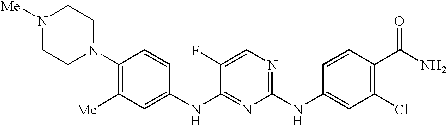 |

Although the foregoing invention have been described in some detail to facilitate understanding, it will be apparent that certain changes and modifications may be practiced within the scope of the appended claims. Accordingly, the described embodiments are to be considered as illustrative and not restrictive, and the invention is not to be limited to the details given herein, but may be modified within the scope and equivalents of the appended claims.

All literature and patent references cited throughout the application are incorporated by reference into the application for all purposes.

What is claimed is:

1. A method of inhibiting an activity of a JAK kinase, comprising contacting the JAK kinase with an amount of a JAK-selective inhibitory 2,4-pyrimidinediamine compound effective to inhibit an activity of the JAK kinase, wherein said JAK-selective inhibitory 2,4-pyrimidinediamine has an IC₅₀ of Syk inhibition of at least about 50 μM.

2. The method of claim 1 in which the JAK-selective inhibitory 2,4-pyrimidinediamine compound is selected from a compound illustrated in TABLE 1.

3. The method of claim 2 which is carried out in vivo.

4. A method of inhibiting an activity of a JAK kinase, comprising contacting in vitro a JAK3 kinase with an amount of a JAK-selective inhibitory 2,4-pyrimidinediamine compound effective to inhibit an activity of the JAK kinase, wherein said JAK-selective inhibitory compound is at least about 10-fold selective for JAK kinase as compared to Syk kinase.

5. The method of claim 4 in which the JAK-selective inhibitory 2,4-pyrimidinediamine compound is at least about 100-fold selective for JAK kinase as compared to Syk kinase.

6. The method of claim 5 in which the JAK-selective inhibitory 2,4-pyrimidinediamine compound is selected from a compound illustrated in TABLE 1 or TABLE 2.

7. The method of claim 4 in which the JAK kinase is an isolated JAK kinase.

8. The method of any one of claim 1 in which the JAK activity inhibited is phosphorylation of a JAK substrate by activated JAK.

9. A method of treating a T-cell mediated autoimmune disease, comprising administering to a patient suffering from such an autoimmune disease an amount of a JAK-selective inhibitory 2,4-pyrimidinediamine compound effective to treat the autoimmune disease.

10. The method of claim 9 in which the autoimmune disease is selected from multiple sclerosis (MS), psoraisis, and Sjogran's syndrome.

11. The method of claim 9 in which the JAK-selective inhibitory 2,4-pyrimidinediamine compound is selected from a compound illustrated in TABLE 1 or TABLE 2.

12. The method of claim 11 in which the JAK-selective inhibitory compound is administered in combination with, or adjunctively to, a compound that inhibits Syk kinase with an IC₅₀ in the range of at least 10 μM.

13. A method of treating an immune-related disease in a patient that is non-responsive to treatment with an inhibitor of Syk kinase, comprising administering to the patient an amount of a JAK-selective inhibitory 2,4-pyrimidinediamine compound to treat the immune disease.

14. The method of claim 13 in which the JAK-selective inhibitory 2,4-pyrimidinediamine compound is selected from a compound illustrated in TABLE 1 or TABLE 2.

15. A method of treating or preventing allograft transplant rejection in a transplant recipient, comprising administering to the transplant recipient an amount of a JAK-selective inhibitory 2,4-pyrimidinediamine compound effective to treat or prevent the rejection.

16. The method of claim 15 in which the rejection is acute rejection.

17. The method of claim 15 in which the rejection is chronic rejection.

18. The method of claim 15 in which the rejection is mediated by the HVGR.

19. The method of claim 15 in which the allograft transplant is selected from a kidney, a heart, a liver and a lung.

20. The method of any one of claim 15 in which the JAK-selective 2,4-pyrimidinediamine compound is at least about 10-fold selective for JAK kinase as compared to Syk kinase.

21. The method of claim 20 in which the JAK-selective inhibitory 2,4-pyrimidinediamine compound is at least about 100-fold selective for JAK3 kinase as compared to Syk kinase.

22. The method of claim 21 in which the JAK-selective inhibitory 2,4-pyrimidinediamine compound is at least about 200-fold selective for JAK3 kinase as compared to Syk kinase.

23. The method of any one of claim 15 in which the JAK-selective inhibitory 2,4-pyrimidinediamine compound is selected from a compound illustrated in TABLE 1 or TABLE 2.

24. The method of claim 23 in which the JAK-selective inhibitory 2,4-pyrimidinediamine compound is administered in combination with, or adjunctively to, another immunosuppressant.

25. The method of claim 24 in which the immunosuppressant is selected from cyclosporine, tacrolimus, sirolimus, an inhibitor of IMPDH, mycophenolate, mycophanolate mofetil, an anti-T-Cell antibody and OKT3.

26. The method of claim 24 in which the JAK3-selective inhibitory 2,4-pyrimidinediamine compound is administered in combination with, or adjunctively to, a compound that inhibits Syk kinase with an IC₅₀ of at least 10 μM.

27. A method of treating or preventing a Type I hypersensitivity reaction, comprising administering to a subject an amount of a JAK-selective inhibitory 2,4-pyrimidinediamine compound effective to treat or prevent the hypersensitivity reaction, wherein the JAK-selective inhibitory 2,4-pyrimidinediamine compound has an IC₅₀ of Syk inhibition of at least about 50 μM.

28. The method of claim 27 in which the JAK-selective inhibitory 2,4-pyrimidinediamine compound is selected from a compound illustrated in TABLE 1.

29. The method of claim 27 which is practical prophylactically, and the JAK-selective inhibitory 2,4-pyrimidinediamine compound is administered prior to exposure to an allergen.

30. A method of inhibiting a signal transduction cascade in which JAK3 kinase plays a role, comprising contacting a cell expressing a receptor involved in such a signaling cascade with a JAK-selective inhibitory 2,4-pyrimidinediamine compound that has an IC₅₀ of Syk inhibition of at least about 50 μM.

31. The method of claim 30 in which the JAK-selective inhibitory 2,4-pyrimidinediamine compound is selected from a compound illustrated in TABLE 1.

32. A method of treating or preventing a JAK kinase-mediated disease, comprising administering to a subject an amount of JAK-selective inhibitory 2,4-pyrimidinediamine compound that has an IC₅₀ of Syk inhibition of at least about 50 μM.

33. The method of claim 32 in which the JAK-selective inhibitory 2,4-pyrimidinediamine compound is selected from a compound illustrated in TABLE 1.

34. The method of claim 33 in which the JAK-mediated disease is the HVGR.

35. The method of claim 33 in which the JAK-mediated disease is acute allograft rejection.

36. The method of claim 33 in which the JAK-mediated diseases is chronic allograft rejection.
